(19) United States
(12) Patent Application Publication
(10) Pub. No.: US 2010/0286680 A1
(43) Pub. Date: Nov. 11, 2010

(54) ABLATION APPARATUS

(76) Inventor: Gennady Kleyman, Brooklyn, NY (US)

Correspondence Address: THE FARRELL LAW FIRM, LLP, 290 Broadhollow Road, Suite 210E, Melville, NY, 11747, US

(21) Appl. No.: 12/516,331

(22) PCT Filed: Nov. 26, 2007
(86) PCT No.: PCT/US07/24399

§ 371 Date: Mar. 8, 2010

(30) Foreign Application Priority Data
Nov. 24, 2006 (US) 11603866
Nov. 26, 2007 (US) 11944974

(51) Int. Cl. A61B 18/04

(52) U.S. Cl. 606/28

(57) ABSTRACT

Disclosed is an apparatus for ablating biological tissues, the apparatus is configured with a cannula, a balloon inflatable with a gaseous medium and coupled to the cannula, and an electromagnetic antenna coupled to the balloon operative to emit electromagnetic waves which heat the wall of the balloon. The wall is made from wave penetrating material impregnated with a plurality of wave absorbing particles which are heated to the desired ablation temperature by the absorbed electromagnetic waves.

### PRIORITY

This application is a continuation-in-part application that claims priority to U.S. patent application Ser. No. 11/603,866 and patent application Ser. No. 11/994,974, filed Nov. 24, 2006 and Nov. 26, 2007, respectively, the disclosure of each of which is incorporated herein by reference.

### BACKGROUND OF INVENTION

1. Field of the Invention

The invention relates to an electromagnetic apparatus for ablating biological tissues.

2. Description of the Related Art

Conventional medical devices used for thermal ablation operate by applying heat, either directly or indirectly, to diseased biological tissues. At least some conventional devices insert inflatable balloons into a cavity of a patient's body. Such conventional devices for ablating biological tissue typically utilize a liquid to inflate the balloon after the device is inserted into the cavity. Liquid within the balloon is then heated to operative temperature and for a period of time sufficient to cause the ablation of tissue. See, e.g. U.S. Pat. Nos. 5,843,144, 5,902,251, 6,041,260, 6,366,818 and 6,447,505, the contents of each of which is incorporated by reference herein.

Conventional devices typically utilize liquids function to store, deliver and conduct heat energy. Liquids used in for conventional devices typically reach a boiling point at temperatures somewhat higher than 70° C. for water or water-based solutions and 195° C. for glycerin. However, heating the liquid to around the boiling point causes gasification of the liquid in the balloon and results in uneven distribution of heat transferred through the balloon's periphery, since gases and liquids have different values of thermal conductivity. As a result, a region or regions of diseased tissue may be inadequately ablated, while healthy tissues may be detrimentally heated. Accordingly, conventional devices are configured to prevent generating heat above the boiling temperature. Clearly, utilizing liquids as a heat-conductive element in an ablation apparatus is associated with undesirable heat-distribution effects that may lead to serious medical complications or inadequately performed surgeries. It is well known that liquids, for example, water or saline solution, have high specific heat values. When energy from an external power source is absorbed in a liquid at a fixed rate, the rate of temperature increase is necessarily small, according to well-known, fundamental thermodynamic principles. This fact increases the time required to attain a therapeutic temperature during a treatment. During prolonged heat exposure time, the heat transfers from treated diseased tissues to neighboring healthy tissues and may inadvertently damage the latter.

It is not unusual for an inflatable balloon to rupture while inside a body cavity during a treatment. The thermal capacity of a liquid in the balloon is relatively large. If a relatively hot liquid is inadvertently released from the balloon into the body cavity, not only may it damage a layer of healthy tissues in contact with the balloon, but its heat energy also may penetrate at a substantial depth into the layers of tissue underlying both the healthy and diseased tissue layers. As a consequence, the balloon inflatable by a liquid may cause serious medical hazards.

Furthermore, the regions of diseased tissue to be ablated are typically localized and thus are relatively small compared to the entire area of healthy biological tissue which is juxtaposed with an inflatable balloon. Consequently, heating the entire periphery of the balloon is usually unnecessary and, again, may be hazardous to a large region of healthy tissue. A need therefore exists in configuring the balloon with selectively heatable peripheral regions, i.e. a wall, to target the regions of diseased tissue while minimizing heating the healthy tissue.

It is, therefore, desirable to provide an apparatus for thermally treating a biological tissue that allows for a relatively brief treatment in a safe and target-oriented manner.

It is also desirable to provide an apparatus for thermally treating a biological tissue by utilizing a gaseous medium as a fluid with a small specific heat to fill a balloon.

It is further desirable to provide an apparatus for thermally treating a biological tissue that is powered by an electromagnetic energy source to transmit energy through a gaseous medium to minimize a period of time necessary for reaching the desirable temperature.

It is also further desirable to provide an apparatus for thermally treating a biological tissue that has an inflatable balloon configured with selective electromagnetically-energy-absorbing areas to target diseased tissues while minimizing heat exposure of healthy tissues.

### SUMMARY OF THE INVENTION

The present invention addresses at least the above-described problems and/or disadvantages and provides at least the advantages described below. Accordingly, an aspect of the present invention provides a method and apparatus for ablation by selectively heating a biological tissue in a cavity so as to minimize exposure of a healthy tissue to heat. In a preferred embodiment, the apparatus is configured with a cannula provided with a body shaped and dimensioned to penetrate a cavity in a body of a patient and with an inflatable balloon coupled to the body and configured to thermally treat a diseased tissue in the cavity. The apparatus further has an antenna or applicator coupled to the cannula and excitable to radiate electromagnetic waves that propagate or otherwise are transmitted through a fluid in the balloon. In the description of the present invention, a fluid includes liquid and gas.

According to one aspect, the inventive apparatus operates with a gaseous medium filling the inflatable balloon and with an electromagnetic power source. The use of the gaseous medium and electromagnetic energy accelerates heating at least a portion of the balloon's peripheral wall, which is impregnated with particle fillers that cause electromagnetic energy to be almost entirely absorbed. The operation of this apparatus leaves the low density and non-absorbing gaseous medium practically thermally unaffected. As a result, the risk of thermal damage of the biological tissue, if and when the balloon ruptures or leaks, is minimized. In contrast, of course, if the balloon were filled with liquid, as disclosed in conventional devices, the latter would absorb heat and, if the balloon ruptures, the heated liquid may damage a large, deep region of biological tissue. In accordance with a further aspect of the invention, the wall of the balloon is configured to be selectively heated to a predetermined temperature for thermally treating the diseased tissue, while neighboring regions of the wall remain unheated. This is achieved by providing the wall of the balloon, which allows electromagnetic waves to penetrate therethrough, with at least one wall region in which wave penetrating material is impregnated with wave absorbing particles or fillers. At the same time, the regions of the wall which are free from the heat absorbing particles remain substantially thermally unaffected. As a result, upon inserting the balloon into a cavity, the heat absorbing region or regions of the balloon juxtaposed with diseased tissues provide effective thermal treatment of the targeted diseased tissues.

The above and other features and advantages of the disclosed apparatus are described hereinbelow in conjunction with the following drawings.

### BRIEF DESCRIPTION OF THE DRAWINGS

The above and other features and advantages of exemplary embodiments of the present invention will be more apparent from the following detailed description taken in conjunction with the accompanying drawings, in which:

FIG. 1 is a view of the inventive thermal ablation apparatus in accordance with a preferred embodiment of the invention;

FIG. 2 is a cross-sectional view of a handpiece shown in FIG. 1;

FIG. 3 is a side elevational view of an inflatable balloon shown in FIG. 2;

FIG. 4 is a side elevational view of the balloon of FIG. 2;

FIG. 5 is a side elevational view of the apparatus of FIG. 2 having a cannula and an antenna configured in accordance with a further embodiment of the invention;

FIG. 6 is a side elevational view of the apparatus of FIG. 5 illustrating a further embodiment of the invention;

FIG. 7 is an enlarged cross-sectional view of an inlet fluid port provided in the handpiece of FIG. 2 and in flow communication with the fluid supply system of FIG. 1;

FIG. 8 is an enlarged cross-sectional view of an outlet fluid port of the handpiece of FIG. 2 in flow communication with the inlet port and opening into the inflatable balloon; and

FIG. 9 is a schematic view of power and fluid supply and control systems.

### DETAILED DESCRIPTION OF THE PREFERRED EMBODIMENTS

Reference will now be made in detail to several views of the invention that are illustrated in the accompanying drawings. Wherever possible, same or similar reference numerals are used in the drawings and the description to refer to the same or like parts or steps. The drawings are in simplified form and are not to precise scale. For purposes of convenience and clarity, directional terms, such as rear and front may be used with respect to the drawings. These and similar directional terms should not be construed to limit the scope of the invention in any manner. The words “connect,” “couple,” and similar terms with their inflectional morphemes do not necessarily denote direct and immediate connections, but also include connections through mediate elements or devices.

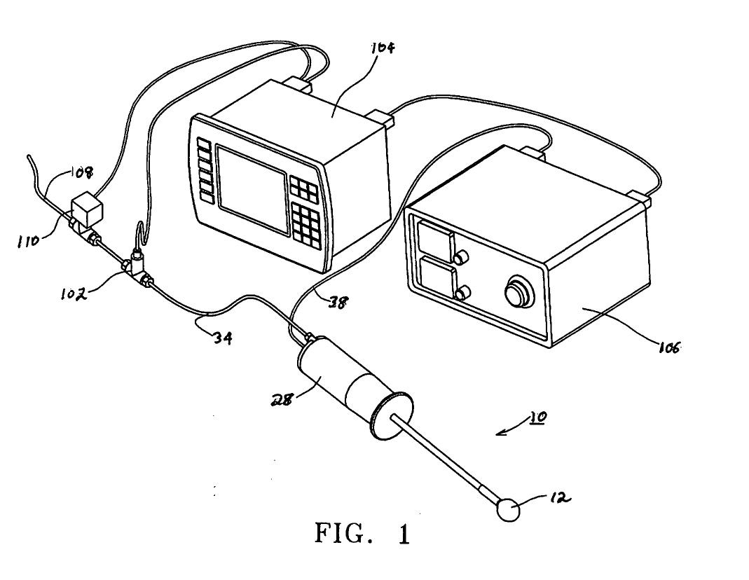

FIG. 1 provides an overall view of an electromagnetic apparatus for ablation configured in accordance with a preferred embodiment of the invention and operative to perform a minimally invasive surgery associated with a thermal treatment of biological tissues in general and, in particular, endometrial ablation. A cannula 10, shaped and dimensioned for introduction into a cavity, includes an inflatable balloon 12. The balloon 12 receives pressurized fluid, such as air or other gaseous medium, through a pneumatic supply line 34 and expands to the desired position. An electromagnetic generator 106 is coupled to an antenna 14 located within balloon 12 via conductive elements or wires 38. In a preferred embodiment, generator 106 is an oscillator that provides a pulsating energy beam. Oscillator 106 can be either a microwave or electromagnetic (radio frequency) oscillator in preferred embodiments of the invention.

When excited, antenna 14 emits energy waves that propagate through the gaseous medium and are selectively absorbed by the wall of balloon 12, causing wave absorbing wall regions to heat, whereas wave penetrating wall regions remain substantially thermally unaffected. The temperature and pressure control of fluid are monitored by a control unit 104 operating a pressure transducer 110 and a valve 102 in a manner discussed hereinbelow. In a preferred embodiment in which the energy was is transmitted through a gaseous medium, oscillator 106 provides for rapid heating of the wave absorbing regions of balloon 12, effectively ablating the diseased tissue in a time-effective, safe operation.

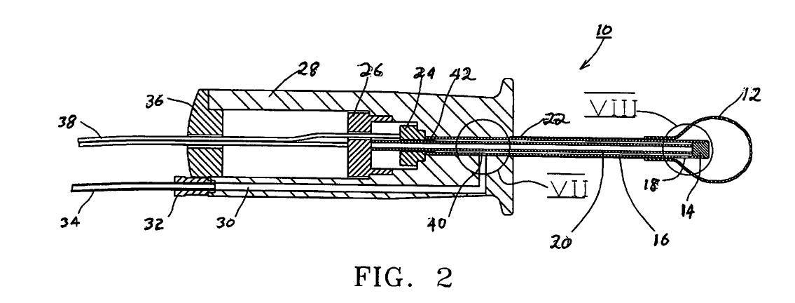

Referring to FIG. 2, cannula 10 is configured with an elongated body 28 made from a heat-insulating material, such as a plastic. The rear or proximal end of body 28 has a cavity closable by a plug 36 which is traversed by wires 38. The wires 38 are coupled to respective elements 26 and 24 which are mounted to the inner surface of body 28 and spaced from plug 36. The elements 24 and 26 are electrically isolated relative to one another and further electrically coupled to respective outer and inner electrodes 16 and 20 which are surrounded by a shield 22 made from heat-shrinking material and circumferentially spaced from one another. The body 28 is provided with distal element 24 and has its distal end sealed to the open end of balloon 12. The distal ends of respective electrodes 16 and 20 are bridged by antenna 14 located within inflatable balloon 12 and operative to energy waves propagating in a gaseous medium within balloon 12. The balloon 12 is made from elastomer, which in a preferred embodiment is silicon impregnated with silver (Ag) and glass fillers. Silicones are generally unaffected by exposure to temperatures reaching 500° F. As a result, those wall regions of balloon 12 that contain only silicone, as described in greater detail below, remain substantially unheated and accordingly do not ablate surrounding biological tissue upon when antenna 14 is excited. In a preferred embodiment, Part #8864-0167-85 provided by Laird Technologies is utilized in consideration of its microwave absorbing properties.

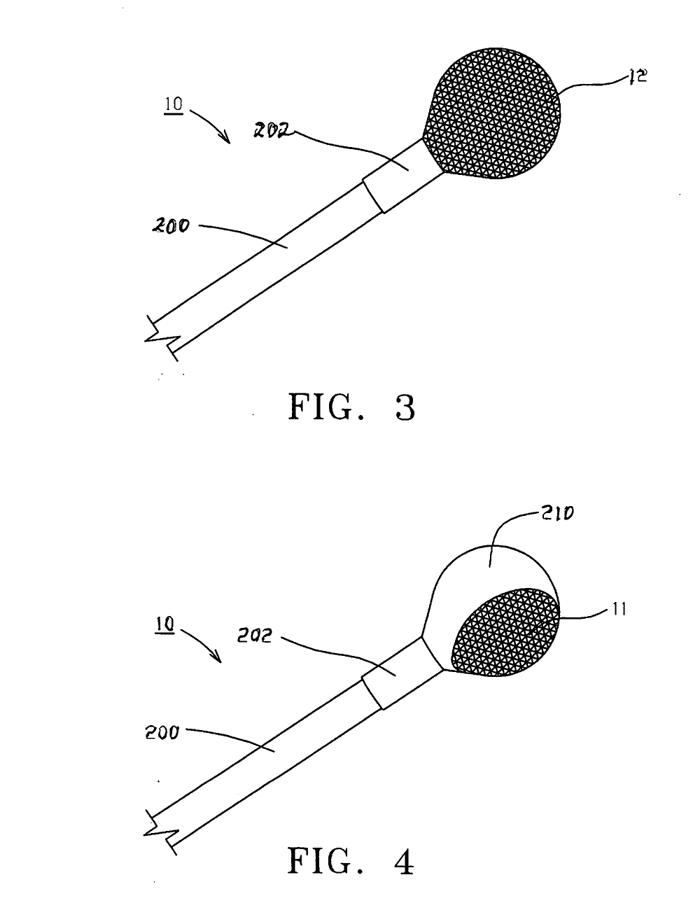

As illustrated in FIG. 3, balloon 12 is attached to a sleeve 202 of cannula 200. To provide heated regions on the wall of the balloon, it is filled with wave-absorbed particles including but not limited to nickel, nickel-plated graphite, silver-plated aluminum, silver-plated copper, silver-plated nickel, silver-plated glass, pure silver, fluorosilicone, fluorocarbon, and ethylene-propylene terpolymer (EPDM). In the embodiment of FIG. 3, these particles are distributed over the entire wall of balloon 12. Thus, electromagnetic waves emitted by antenna 14 propagating through the gaseous medium and further through portions of balloon 12 free from wave-absorbed particles do not substantially thermally affect both. However, impinging upon the particles, electromagnetic energy is transferred into heat energy manifested by heat which is produced by the particles.

Frequently, the tissue to be treated is rather small compared to the entire periphery of balloon 12. Accordingly, providing the wall of balloon 12 with a target oriented wave absorbing region is beneficial in certain embodiments to the patient's health to provide a time-effective ablation or surgery.

As shown in FIG. 4, the wall of balloon 12 has one or more heatable regions 11, which include polymeric material impregnated with wave-absorbing particles elements, and peripheral regions that do not have wave-absorbing elements. The heatable regions 11 are preferably patterned so that when cannula 10 is inserted into the cavity, these regions will be juxtaposed with regions of diseased biological tissue. An electromagnetic antenna in balloon 12 is centered on the longitudinal axis of balloon 12, as shown in FIG. 2, and emits electromagnetic waves, for example microwaves. The electromagnetic waves propagate through a gaseous medium and further penetrate energy-absorbing elements of region or regions 11 enabling, thus, a rapid and high-intensity heat transfer therethrough. On the other hand, the remaining wall of the balloon that lacks fillers remains thermally unaffected by penetrating electromagnetic waves and does not affect a healthy biological tissue, which is juxtaposed with the filler-free wall regions. The region 11 may be variously shaped, dimensioned and located in accordance with target areas containing diseased biological tissues upon inserting cannula 200 into the cavity. In addition to target configured region or regions 11, balloon 12 may be variously shaped and dimensioned to address specific needs of any given patient. Selective shaping provides a further benefit of reducing reflection impact.

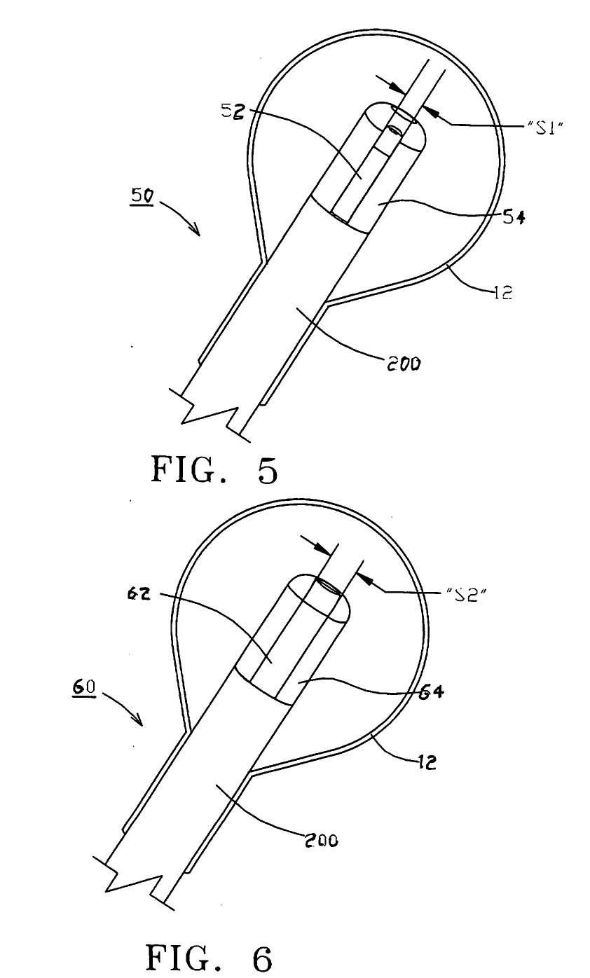

FIG. 5 illustrates a further embodiment of inventive apparatus 50 provided with a cannula 200 which is configured to localize electromagnetic heating of the balloon's periphery. The distal end 54 of cannula 200 has an elongated channel extending generally coaxially with the longitudinal axis of cannula 200 and opening into the distal tip of cannula 200. The channel is shaped and dimensioned to receive antenna 52 with distal end spaced inwards from an open tip of cannula 200. Consequently, when antenna 52 is excited, the waves generally extend along a predetermined path S1, defined by the opening in the tip of the cannula channel, and heat a desired region of balloon 12 juxtaposed with a diseased tissue in the cavity. Although the channel and antenna 52 are shown as being centered about the longitudinal axis of cannula 200, other modifications of the shape of the channel may include bent regions. For example, the channel may have a distal end extending transversely to the longitudinal axis of cannula 200, as shown by dash lines in FIG. 5, and opening into a respective side opening of distal end 54 of cannula 200. The antenna 52 also has its distal end extending transversely to the longitudinal axis along the distal end of the channel. In further preferred embodiments, the configuration of the channel includes multiple transverse passages each having a respective portion of antenna 52.

FIG. 6 illustrates a further modification of inventive apparatus 60 configured with cannula 200 having its distal end 64 machined so as to receive an electromagnetic antenna 62. In contrast to the embodiment shown in FIG. 5, antenna 62 of FIG. 6 has its distal tip lying substantially flush with the outer periphery of the cannula's distal tip. Once antenna 62 is excited, electromagnetic waves, exiting from the opening of the cannula's tip, will generally propagate along a path S2 towards the desired electro-conductive region of the balloon's periphery. Since the desired target region of balloon 12 is preferably juxtaposed with a diseased tissue, the latter will be effectively thermally treated. Meanwhile, the remaining periphery of balloon 12 is minimally thermally affected and thus does not ablate healthy biological tissues.

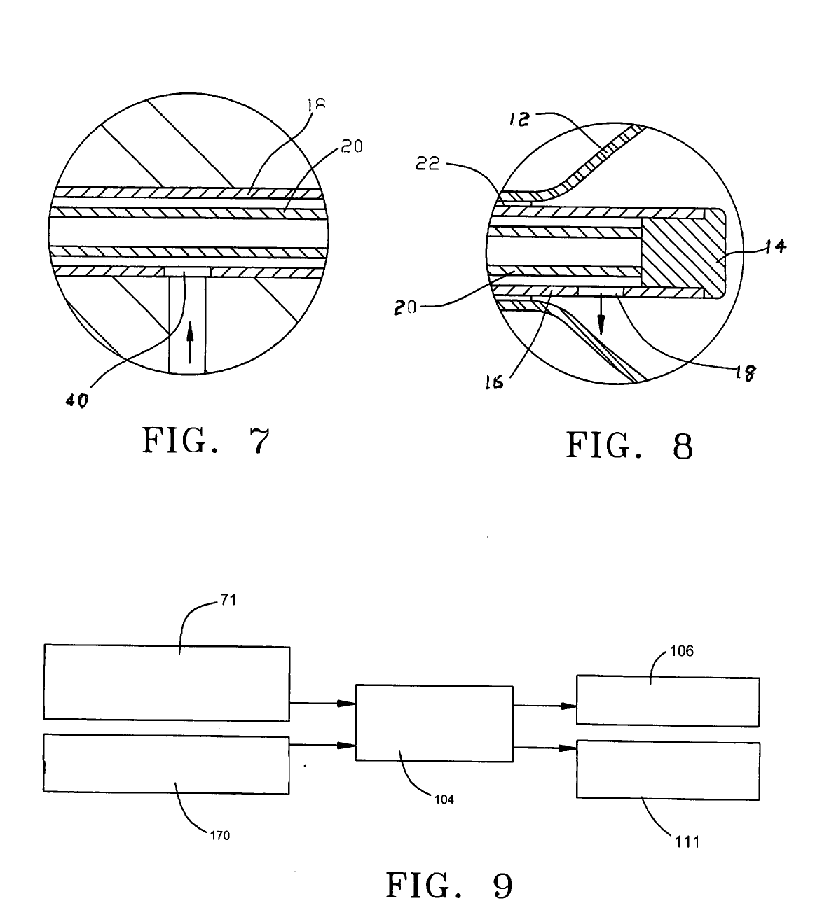

Turning to FIGS. 2, 7 and 8, body 28 is provided with an offset channel 30 which is sealingly coupled to pneumatic supply line 34 by a sealing element 32 so that supply line 34 and channel 30 are in flow communication. The channel 30 extends generally parallel to the longitudinal axis of body 28 and has a distal end extending transversely to the longitudinal axis and opening into an inlet port 40 of body 28 in the vicinity of the distal end of body 28. Upon traversing port 40, fluid is further advanced along body 28 towards an outlet port 18 located within balloon 12, as illustrated in FIG. 4. As the fluid enters the balloon 12, the latter expands filling the patient's cavity.

Referring to FIGS. 1 and 9, in operation, the apparatus is inserted into the patient's cavity and the pressurized gas from a fluid pressurizing device 111 is supplied to inflatable balloon 12, which causes the balloon to expand and fill the treated cavity. The required level of pneumatic pressure is determined by controller 104 and monitored by pressure transducer 110. The electromagnetic generator 106 is then energized to excite antenna 14 through wires 38. The antenna 14 produces waves in a pre-selected range, which are absorbed by the wave-absorbing particles of the elastomeric material in the wall of inflatable balloon 12. The electromagnetic energy absorbed by the wave-absorbing particles is transformed into heat energy, which causes the ablation of the treated tissue. The level of temperature sufficient to cause the ablation and the time required to reach this temperature are determined by the amount of electromagnetic energy produced by the electromagnetic generator and the density of the wave-absorbing particles in the conductive elastomeric material. Generally, the level of the generated electromagnetic energy is selected to reach the maximum ablation temperature in a shortest period of time, in order to reduce the time of treatment and thus prevent or minimize the undesirable heat transfer from treated diseased tissue to neighboring healthy tissue. A temperature sensor 71 is operative to monitor a temperature of the balloon periphery and coupled to controller 104, which, in turn, is operative to control power source 106 so as to maintain the desired temperature.

In case of rupture of balloon 12 or a sudden cavity contraction, the pressure inside inflatable balloon 12 may go outside of the range preset in controller 104. In such a case, the pressure transducer 110 provides feedback of the pressure change to the controller 104, to control and shut off the pneumatic pressurizing device 111 and electromagnetic generator 106 if necessary.

Specific features described herein may be used in some embodiments, but not in others, without departure from the spirit and scope of the invention as set forth. Although operating the inventive apparatus in a microwave range may be preferred, other electromagnetic wave lengths can be successfully utilized within the scope of the invention. The disclosed apparatus can be used in a variety of surgeries including, for example, endometrial ablation.

While the invention has been shown and described with reference to an exemplary embodiment thereof, it will be understood by those skilled in the art that various changes in form and details may be made therein without departing from the spirit and scope of the invention.

1. An apparatus for ablating biological tissues, the apparatus comprising:
a cannula configured to penetrate into a cavity of a patient;
an inflatable balloon coupled to the cannula; and
a wall of the balloon, wherein the wall includes a plurality of energy absorbing particles.

2. The apparatus of claim 1, wherein the plurality of energy absorbing particles are spaced apart over an entire surface of the wall of the balloon.

3. The apparatus of claim 1, further comprising an antenna positioned adjacent to the balloon to heat the plurality of energy absorbing particles.

4. The apparatus of claim 1, further comprising an antenna positioned within the balloon to heat the plurality of energy absorbing particles.

5. The apparatus of claim 1, wherein heating the balloon elevates the balloon to a predetermined temperature for ablating biological tissue.

6. The apparatus of claim 1, wherein the wall of the balloon comprises internal and external layers, with the internal layer including the plurality of energy absorbing particles distributed to absorb electromagnetic energy.

7. The apparatus of claim 6, wherein the external layer is made from a biologically inert material, and wherein the external layer is configured to directly contact the cavity when the cannula penetrates the cavity.

8. (canceled)

9. (canceled)

10. The apparatus of claim 1, wherein energy is provided by one or more of radio frequency energy, microwave energy, millimeter waves and laser.

11. The apparatus of claim 10, wherein the energy is provided by pulsation of applied electromagnetic energy, and
the plurality of energy absorbing particles are manufactured from one or more of nickel, nickel-plated graphite, silver-plated aluminum, silver-plated copper, silver-plated nickel, silver-plated glass and pure silver.

12. The apparatus of claim 1, further comprising:
a pneumatic line coupled to the cannula for supplying a gaseous medium for inflating the balloon; and
an antenna coupled to the cannula and extending into the inflatable balloon, the antenna configured to propagate energy through the gaseous medium for absorption by the plurality of energy absorbing particles.

13. (canceled)

14. The apparatus of claim 12, wherein the plurality of energy absorbing particles are clustered to define an absorption wall region of the balloon and a penetrating wall region, wherein the absorption region absorbs electromagnetic energy and the penetrating region is substantially thermally unaffected by electromagnetic energy.

15. The apparatus of claim 14, wherein the balloon is configured to have at least one or more absorbing wall regions configured to oppose one or more corresponding regions of diseased biological tissue, upon penetrating the cavity.

16. The apparatus of claim 3, wherein a distal end of the cannula has a channel configured to receive the antenna, wherein the channel opens into the balloon so that energy propagates towards a wall region of the balloon substantially aligned with the channel for heating the wall region to ablate diseased biological tissue, without substantial heating of other regions of the balloon.

17. The apparatus of claim 16, wherein a distal end of the antenna is inwardly spaced from the distal end of the channel, wherein the distal end of the antenna and the distal end of the cannula are flush.

18. (canceled)

19. The apparatus of claim 3, further comprising:
a power source for exciting the antenna;
an electro-conductive element coupling the power source to the antenna and extending to the cannula; and
a pressurizing device for pressurizing the balloon via a fluid path through the cannula.

20. An apparatus for thermal treatment of biological tissues, the apparatus comprising:
a guidable cannula for penetrating a cavity of a patient;
an inflatable balloon sealingly coupled to the cannula; and
an antenna coupled to the cannula and terminating in the balloon, wherein the antenna is excited to propagate energy through a gaseous medium in the balloon to selectively heat one or more wall regions of the balloon.

21. The apparatus of claim 20, wherein the cannula is configured with a channel in flow communication with a conduit having an outlet port opening into the balloon so that fluid traversing the outlet port inflates and urges the balloon against a surface of the cavity.

22. The apparatus of claim 21, further comprising:
a pressure transducer in flow communication with the conduit for monitoring pressure of the gaseous medium in the balloon;
a temperature transducer for monitoring temperature at the outer wall of the balloon; and
a control unit receiving signals from the pressure and temperature transducers for controlling a power source and pressure of the gaseous medium in the balloon.

23. A method for thermally treating biological tissues, the method comprising:
penetrating, utilizing a cannula having an inflatable balloon coupled thereto, a cavity of a patient; and
heating a plurality of energy absorbing particles positioned on a wall region of the balloon.

24. The method of claim 23, wherein the heating is provided by energizing an antenna positioned adjacent to the balloon.
